(19) United States
(12) Patent Application Publication
(10) Pub. No.: US 2013/0321773 A1
(43) Pub. Date: Dec. 5, 2013

(54) Optometric Automatic Test Device and Method

(71) Applicants: Cheng-Chung Lee, Tainan (TW); Yu-Ting Li, Tainan (TW); Wen-Hong Wu, Tainan (TW); Chun-Li Chang, Tainan (TW)

(72) Inventors: Cheng-Chung Lee, Tainan (TW); Yu-Ting Li, Tainan (TW); Wen-Hong Wu, Tainan (TW); Chun-Li Chang, Tainan (TW)

(21) Appl. No.: 13/488,623

(22) Filed: Jun. 5, 2012

(51) Int. Cl. A61B 3/10

(52) U.S. Cl. 351/211

(57) ABSTRACT

An optometric automatic test device and method for a lens are provided to perform image capturing and reading with an image analysis technique, perform automatic focusing by automated control technology rather than by the test worker's operation, and enable two tests, namely refractive power test and astigmatism test, to be performed on eyeglass lenses automatically, so as to greatly reduce the errors caused by the test worker's operation and enhance test efficiency.

### BACKGROUND OF THE INVENTION

1. Technical Field

The present invention relates to optometric automatic test devices and methods, and more particularly, to an optometric automatic test device and method for testing the refractive power and astigmatism of a lens.

2. Description of Related Art

Recently, in Taiwanese people's eyes, eyeglasses are becoming less functional but more like personal add-on. Hence, there is a surge of the demand for various eyeglasses. However, the market is rife with a wide variety of eyeglasses, such as vision correction eyeglasses, protective eyeglasses, and sunglasses, whose quality varies. Hence, eyeglasses are usually subjected to several tests in terms of refractive power, astigmatism, diffused light, prism diopter, visual field, polarized test, etc. Among the aforesaid tests, refractive power and astigmatism have a marked effect on whether an eyeglasses wearer is able to see clearly, and thus they are important lens quality tests indispensable to various protective eyeglasses. Referring to FIG. 1, there is shown a schematic view of the framework of a system of refractive power and astigmatism optometric test instruments by international standard ANSI/ISEA Z87.1-2010[1]. As shown in FIG. 1, the test system comprises an eyepiece B, a manual focus-adjustment mechanism C, a telescope D, a subject lens fixing mechanism E, a test pattern F, and a light box G, which are required for a test worker's eye A to perform observation.

To perform the refractive power test or the astigmatism test, it is necessary for the test worker to observe the solar test pattern F on the light box G with eyepiece B in conjunction with the manual focus-adjustment mechanism C, such that lines on the solar test pattern F are presented in the most well-defined manner, and then the test worker records the focus adjustment position as a reference for calculating the refractive power or astigmatism. The result of the aforesaid test procedure depends on observation performed with the human eyes. Vision is different from person to person, and so is observation-related behavior. As a result, not only is there a difference in the results between the tests performed by different test workers on the same test system and the same subject lens E, but the same test worker yields different results at different points in time (for example, in the morning versus in the afternoon.)

When watching for a long period of time, the human eye is likely to get tired to the detriment of precision in the measurement that has been being carried out for a long period of time. Manual operation is inevitably susceptible to errors; hence, in practice, a lens test is usually performed by multiple test workers in multiple instances repeatedly in order to reduce operation-induced errors, albeit at the cost of a lengthy test and high test costs.

### SUMMARY OF THE INVENTION

The present invention provides an optometric automatic test device and method for a lens, regarding two tests, namely refractive power test and astigmatism test. The objective of the present invention is to perform image capturing and reading with an image analysis technique, perform automatic focusing by automated control technology rather than by the test worker's operation, and enable two tests, namely refractive power test and astigmatism test, to be performed on eyeglass lenses automatically, so as to greatly reduce the errors caused by the test worker's operation and enhance test efficiency.

The present invention is directed to an optometric automatic test device and method for a lens. The optometric automatic test device for a lens comprises a base, an axial movable platform disposed on the base, an image sensing unit, a rear barrel, an optical zoom lens, a test pattern, an illumination unit, and a control unit.

The base enables axial angular adjustment of the optical zoom lens and radial angular adjustment of the optical zoom lens so as to align the center of the test pattern with the center of the image sensing unit. The image sensing unit is connected to the rear end of the rear barrel for receiving an image from the optical zoom lens. The optical zoom lens comprises at least a lens and a lens barrel. The movable platform and the rear barrel are connected, such that the positions of an image sensor and the optical zoom lens relative to each other change simultaneously in response to displacement of the movable platform. The test pattern is positioned in front of the optical zoom lens. The illumination unit is positioned behind the test pattern, such that the pattern displays an image with excellent contrast in brightness. A subject lens is disposed between the optical zoom lens and the test pattern.

The optometric automatic test method for a lens is intended to measure three parameters, namely refractive power, astigmatism, and visual acuity.

The present invention provides an optometric automatic test device and method for a lens to perform image capturing and reading with an image analysis technique, perform automatic focusing by automated control technology rather than by the test worker's operation, and enable two tests, namely refractive power test and astigmatism test, to be performed on eyeglass lenses automatically, so as to greatly reduce the errors caused by the test worker's operation and enhance test efficiency.

### BRIEF DESCRIPTION OF THE SEVERAL VIEWS OF THE DRAWINGS

FIG. 1 (PRIOR ART) is a system structural schematic view of a conventional optometric test instrument;

FIG. 2 is a system structural schematic view of an optometric automatic test device according to the present invention;

FIG. 3 is a perspective view of the optometric automatic test device according to the present invention;

FIG. 4 is a graph of contrast evaluation value against focusing position of a movable platform according to an embodiment of the present invention;

FIG. 5 is a partial enlarged view of the graph in FIG. 4 according to an embodiment of the present invention;

FIG. 6 is a schematic view of astigmatism measurement of contrast evaluation value measurement area according to an embodiment of the present invention;

FIG. 7 are graphs of contrast against focusing position at different angles of a solar test pattern based on the result of astigmatism measurement according to an embodiment of the present invention;

FIG. 8A is a schematic view of the minimum refractive power position of an astigmatism measurement capturing image according to an embodiment of the present invention;

FIG. 8B is a schematic view of the average refractive power position of an astigmatism measurement capturing image according to an embodiment of the present invention;

FIG. 8C is a schematic view of the maximum refractive power position of an astigmatism measurement capturing image according to an embodiment of the present invention;

FIG. 9A is a schematic view of a lens-free acuity sample image according to an embodiment of the present invention; and

FIG. 9B is a schematic view of a subject lens-enabled acuity sample image according to an embodiment of the present invention.

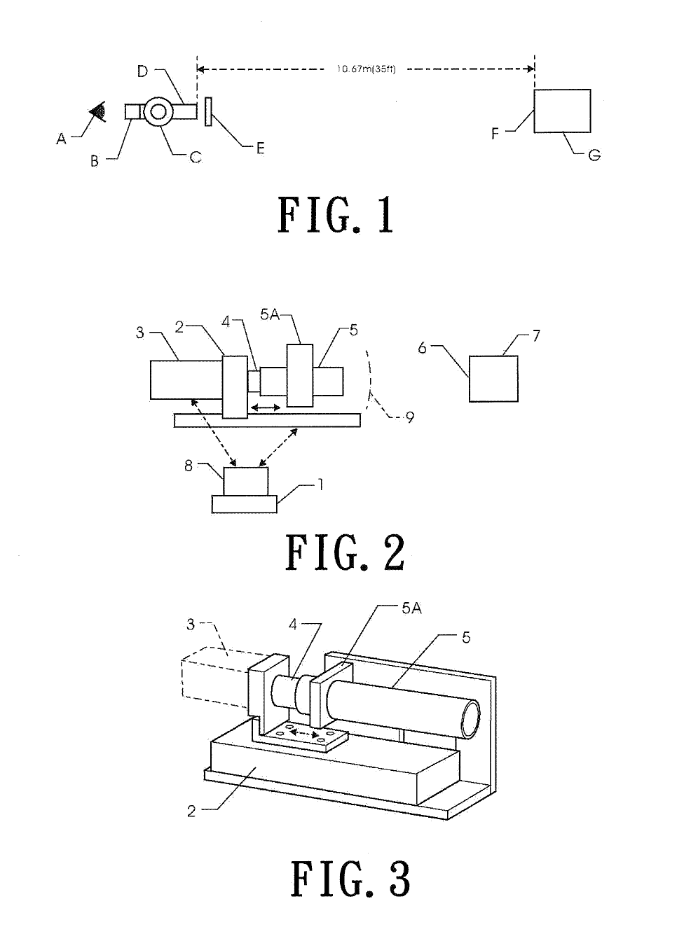

### DETAILED DESCRIPTION OF THE EMBODIMENTS OF THE INVENTION

The present invention provides an optometric automatic test device and method for use with a lens. Referring to FIG. 2 and FIG. 3, there are shown a structural schematic view and a perspective view of an optometric automatic test device according to the present invention, respectively. The optometric automatic test device comprises a base 1, an axial movable platform 2 disposed on the base 1, an image sensing unit 3, a rear barrel 4, an optical zoom lens 5, an optical zoom lens holder 5A, a test pattern 6 (such as a solar test pattern), an illumination unit 7, and a control unit 8.

The base 1 enables axial angular adjustment of the optical zoom lens 5 and radial angular adjustment of the optical zoom lens 5 so as to align the center of the test pattern 6 with the center of the image sensing unit 3.

The image sensing unit 3 is connected to the rear end of the rear barrel 4 for receiving an image from the optical zoom lens 5. The optical zoom lens 5 comprises at least a lens and a lens barrel. The movable platform 2 and the rear barrel 4 are connected, such that the positions of the image sensing unit 3 and the optical zoom lens 5 relative to each other change simultaneously in response to displacement of the movable platform 2. The test pattern 6 is positioned in front of the optical zoom lens 5. The illumination unit 7 is positioned behind the test pattern 6, such that the test pattern 6 displays an image with excellent contrast in brightness. A subject lens 9 is disposed between the optical zoom lens 5 and the test pattern 6.

The optometric automatic test method for a lens is intended to measure three parameters, namely (A) refractive power, (B) astigmatism, and (C) visual acuity.

(A) Refractive Power Correction

The two standard lenses are confirmed with two diopter values, respectively. One of the two standard lenses has a positive diopter value, the other one has a negative diopter value. Create the sharpest target image position-related data (Dp, Dn) of the two standard lenses and the lens-free sharpest target image position-related datum (D0). Two lenses of +0.06 D and −0.06 D, respectively, usually function as the two standard lenses with positive and negative diopter values, respectively. Put the subject lens 9 (or eyeglasses) to be evaluated in the system, so that the system automatically determines the sharpest target image position UD and compares UD with the aforesaid Dp, Dn and D0 to calculate the refractive power RP of the subject lens 9. The comparison requires the equation below.

$$RP = \{ \begin{matrix}( UD - {D}_{0} ) \times \frac{- 0.06}{( {D}_{0} - {D}_{n} )} , for UD \leq {D}_{0} \\ ( {D}_{0} - UD ) \times \frac{0.06}{( {D}_{p} - {D}_{0} )} , for UD > {D}_{0}\end{matrix}$$

where RP denotes the refractive power RP of the subject lens 9.

(A) Determining Position of Sharp Target Image

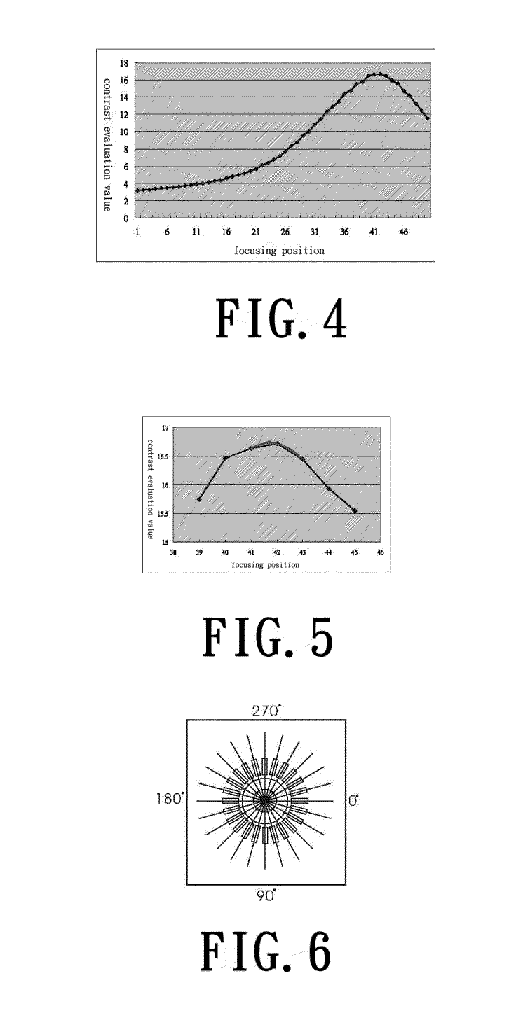

The position of a sharp target image is identified by the maximum value of a contrast index. Referring to FIG. 4, the graph of contrast evaluation value against focusing position of the movable platform 2 is plotted by measuring the contrast evaluation value against the gradual displacement of the movable platform 2 in the same direction. The displacement of the movable platform 2 begins with position 1; and the gradual increase in the contrast value indicates the ongoing improvement in visual acuity. The contrast index value is 16.63 and 16.71 when the movable platform 2 reaches position 41 and 42, respectively. The contrast index value decreases to 16.44 as soon as the movable platform 2 reaches position 43. Hence, the best image sharpness position lies between position 41 and position 43. Referring to FIG. 5, if the distance between position 41 and position 43 meets the precision requirement, position 42 will be selected to be the best image sharpness position. There is likely an overly large error resulting from the position 42 selected to be the best image sharpness position; in such a situation, data fitting can be performed with a quadratic polynomial equation, and then the maximum value position of the quadratic polynomial equation is identified and defined as the best image sharpness position, as shown in FIG. 5.

(B) Lens Astigmatism Measurement

If the subject lens 9 manifests astigmatism, the line sharpness of the solar test pattern 6 will vary from direction to direction. When manual measurement is observed with the naked eye, it is necessary to adjust a focus adjustment knob in order to measure the maximum and minimum refractive powers at which a portion of the lines of the solar test pattern 6 can be sharp. The RP1 and RP2, respectively. The refractive power RP and astigmatism A are calculated with the following equation.

$$RP = \frac{{RP}_{1} + {RP}_{2}}{2} , A = \vert {RP}_{1} - {RP}_{2} \vert \qquad equation ( 2 )$$

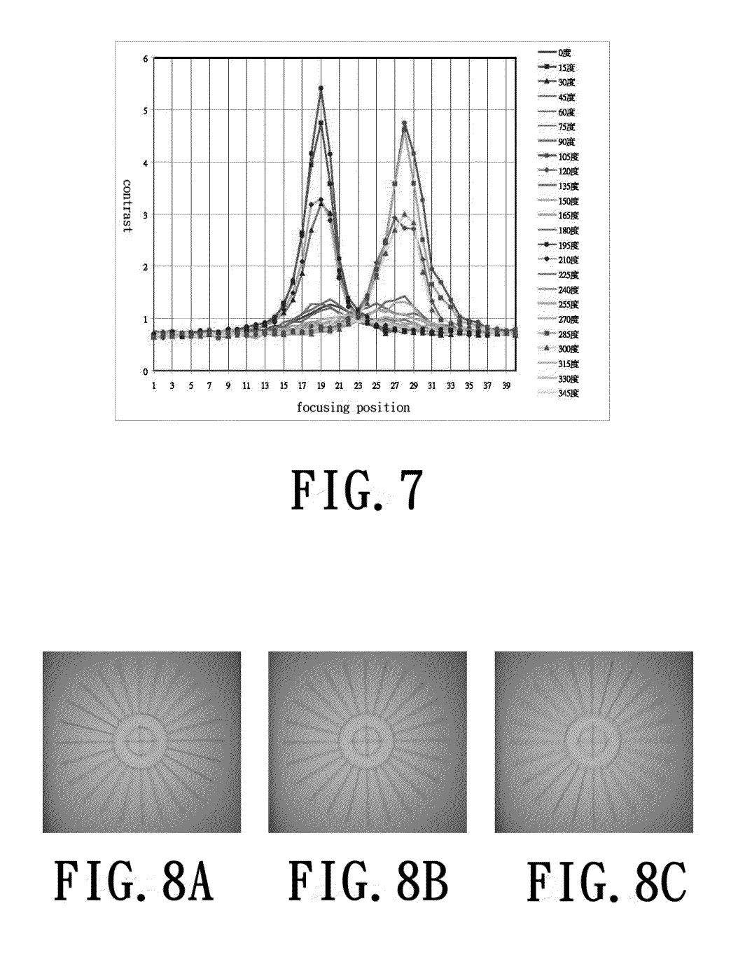

In the presence of astigmatism, the line contrast of the solar test pattern 6 at the same focusing position does not remain unchanged with direction, nor is it possible to predict whether the line contrast in a specific direction will have the maximum or minimum refractive power when focused. Referring to FIG. 7, move for a fixed distance, calculate in the direction of the movement and extensively the line contrast of the lines in all directions, compare the data acquired and related to the line contrast in all directions so as to figure out the maximum and minimum refractive power values, and eventually calculate the refractive power and astigmatism.

Astigmatism measurement is performed by taking the steps of:

1. positioning the subject lens 9 by a test worker;

2. performing image alignment adjustment so as to align the image of the solar test pattern 6 with the center of the alignment reference crosshair on the screen;

3. astigmatism measurement requires taking the steps of:

3a. determining the reference position of astigmatism measurement regarding the automatic focusing of the center of the solar test pattern 6;

3b. moving the movable platform backward to a preset position, moving the movable platform forward step by step and by a small distance, measuring and recording the line contrast of the solar test pattern 6 at each position and in each direction so as to obtain the data shown in FIG. 7; and

4. calculating the refractive power corresponding to the lines of the solar test pattern 6 in all directions, calculating their maximum values RP₁ and minimum values RP₂, and calculating the refractive power RP and astigmatism A with the above equation (2).

(C) Visual Acuity Measurement, Which is Performed by Taking the Steps of:

1. removing all the lenses by the test worker;

2. performing image alignment adjustment to align the image of the solar test pattern 6 (acuity sample) with the center of the alignment reference crosshair on the screen;

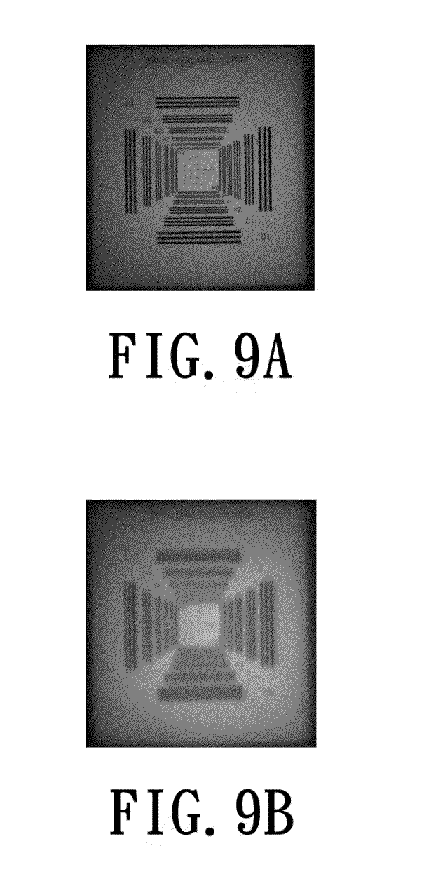

3. capturing a lens-free acuity sample image (as illustrated with FIG. 9A) and recording the visual acuity index value of a user-defined area;

4. positioning the subject lens 9 in place by the test worker;

5. performing image alignment adjustment to align the image of the solar test pattern 6 (acuity sample) with the center of the alignment reference crosshair on the screen;

6. capturing a lens-enabled acuity sample image (as illustrated with FIG. 9B), and recording the visual acuity index value of the user-defined area; and

7. comparing lens-free and lens-enabled visual acuity indices in terms of variation thereof.

The present invention provides an optometric automatic test device and method for a lens to perform image capturing and reading with an image analysis technique, perform automatic focusing by automated control technology rather than by the test worker's operation, and enable two tests, namely refractive power test and astigmatism test, to be performed on eyeglass lenses automatically, so as to greatly reduce the errors caused by the test worker's operation and enhance test efficiency.

What is claimed is:

1. An optometric automatic test device for a lens, comprising:
a base, an axial movable platform disposed on the base, an image sensing unit, a rear barrel, an optical zoom lens, a test pattern, an illumination unit, and a control unit, the base enabling axial angular adjustment of the optical zoom lens and radial angular adjustment of the optical zoom lens so as to align a center of a test pattern with a center of the image sensing unit;
the image sensing unit being connected to a rear end of the rear barrel for receiving an image from the optical zoom lens;
the optical zoom lens comprising at least a lens and a lens barrel;
the movable platform being connected to the rear barrel, such that the positions of an image sensor and the optical zoom lens relative to each other change simultaneously in response to displacement of the movable platform;
the test pattern being positioned in front of the optical zoom lens, the illumination unit being positioned behind the test pattern, such that the pattern displays an image with excellent contrast in brightness; and
a subject lens disposed between the optical zoom lens and the test pattern.

2. An optometric automatic test method for use with a refractive power test, the optometric automatic test method comprising the steps of:
confirming two standard lens with two diopter values, respectively, wherein one of the two standard lenses has a positive diopter value, the other one has a negative diopter value;
creating sharpest target image position-related data (Dp, Dn) of the two standard lens and a lens-free sharpest target image position-related datum (D0); and
putting a subject lens (or eyeglasses) to be evaluated in a test device system, such that the system automatically determines the sharpest target image position UD and compare UD with the aforesaid Dp, Dn and D0 to calculate the refractive power RP of the subject lens.

3. The optometric automatic test method of claim 2, wherein the determining the position of the sharpest target image comprises confirming the position of the sharpest target image from the maximum value of a contrast index, that is, a contrast evaluation value detected while a movable platform is moving in the same direction gradually, so as to determine the best image sharp position.

4. An optometric automatic test method for use with an astigmatism measurement method comprises the steps of:
a. positioning a subject lens by a test worker;
b. performing image alignment adjustment so as to align an image of a solar test pattern with a center of an alignment reference crosshair on a screen;
c. performing the astigmatism measurement comprising the steps of:
c1. determining a reference position of astigmatism measurement regarding automatic focusing of the center of the solar test pattern;
c2. moving a movable platform backward to a preset position, moving the movable platform forward step by step and by a small distance, and measuring and recording line contrast of the solar test pattern at each position and in each direction; and
d. calculating a refractive power corresponding to lines of the solar test pattern in all directions, calculating maximum values RP₁ and minimum values RP₂, and calculating the refractive power RP and astigmatism A.
